(19) United States
(12) Patent Application Publication
(10) Pub. No.: US 2012/0226235 A1
(43) Pub. Date: Sep. 6, 2012

(54) METHOD AND ARRANGEMENT FOR REDUCING AIR BUBBLES IN FLUIDIC SYSTEM

(75) Inventors: Bjørn Gullak Larsen, Birkerod (DK); Steffen Hansen, Hillerod (DK); Kristian Glejbøl, Glostrup (DK); Jens Peter Jensen, Jyllinge (DK)

(73) Assignee: Novo Nordisk A/S, Bagsvaerd (DK)

(21) Appl. No.: 12/673,402

(22) PCT Filed: Aug. 12, 2008
(86) PCT No.: PCT/EP2008/060583

§ 371 Date: Jun. 10, 2010

(30) Foreign Application Priority Data
Aug. 13, 2007 (EP) 07114244.2

(51) Int. Cl. A61M 5/36

(52) U.S. Cl. 604/123

(57) ABSTRACT

The invention provides a fluidic system (100) comprising a permeable membrane (152), in which one side of the permeable membrane is in contact with a fluid, this lowering the partial pressure of a gas on the other side of the membrane being by raising the pressure of vapour from the fluid by diffusion from the fluidic system through the permeable membrane. This will aid in expelling a gas from a fluid assembly as well as reducing the likelihood of gas entering into the fluid assembly.

The present invention generally relates to fluidic systems and methods therefore adapted to reduce problems associated with air bubbles in such systems. In a specific aspect the invention relates to a fluidic device adapted to minimize the size and/or number of air bubbles in a fluid path.

### BACKGROUND OF THE INVENTION

Air bubbles are often a problem in fluidic systems, especially when they are containing chambers interconnected by channels. When filling a system with liquid for the first time, it can be difficult to avoid enclosed air in the system, e.g. in case of dimensional changes of the fluid path. Further, if the fluidic system contains highly permeable elements separating the fluid from the ambient atmosphere, e.g. silicone rubber, air bubbles can also enter into the system by diffusion. This diffusion is driven by differences in partial pressure of the gasses available inside and outside the fluidic system.

When bubbles have been introduced in a fluidic pump system such as a pump, they may cause pressure losses if a bubble filled liquid is transported through a fluid path, especially in the case of dimensional changes in the path. In the case of a piston pump this may result in pump failure on both the inlet and outlet side. A further problem may be varying stroke volumes due to compression of air bubbles.

Bubble problems in a fluidic system may be solved by for example a hydrophobic vent, e.g. a Gore-Tex® membrane placed in the flow path, however, this will work only if the pressure inside the fluid path is higher than the ambient air pressure. Alternatively a bubble trap that prevents the bubbles from entering certain parts of the fluid path may be provided. As this does not eliminate the bubbles, but only separates them from the liquid it takes up a volume to collect the bubbles. Sometimes this volume is not feasible to have in the system.

Having regard to the above problems, it is an object of the present invention to provide a fluidic system adapted to reduce the problems associated with air bubbles trapped in the system during priming. It is a further problem to reduce the problems associated with air bubbles entering into the system through permeable elements separating the fluid from the ambient atmosphere. The system should be reliable in use and simple in design allowing for cost-effective manufacture.

In a specific implementation a membrane pump may be used in combination with a flexible reservoir from which liquid can be sucked through the pump from its inlet to its outlet. An example of a skin-mountable drug delivery device, based on such a combination of a pump and reservoir is shown in WO 2006/077263. In such an arrangement it would be possible to compress the reservoir and thereby force liquid drug through the pump and into the patient carrying the pump, e.g. the patient may stumble or walk into a hard object, or the infusion device may be hit by an object. Although such a flexible reservoir normally will be protected by a relatively rigid housing, the housing may brake when subjected to excessive force, this allowing the flexible reservoir to be compressed and drug thereby unintentionally infused into the patient. To protect against this situation the pump shown in WO 2006/089958 is provided with an effective but relatively complex safety valve.

Having regard to the above-identified problems, it is yet a further object of the present invention to provide a pump assembly comprising a safety valve adapted to prevent unintended flow of fluid through the pump assembly. The pump should provide a high degree of safety of use yet be simple in construction.

### DISCLOSURE OF THE INVENTION

In the disclosure of the present invention, embodiments and aspects will be described which will address one or more of the above objects or which will address objects apparent from the below disclosure as well as from the description of exemplary embodiments. In the context of the present invention the term relative humidity (RH) is used, this being defined as the ratio of the partial pressure of water vapor in a gaseous mixture of air and water to the saturated vapor pressure of water at a given temperature, and expressed as a percentage.

Thus, in a first aspect a fluidic system is provided comprising (a) a fluid assembly comprising a fluid-conducting structure having an inlet and an outlet, first means being permeable to water vapour, and second means being permeable to air, wherein the first and second permeable means have an inner and an outer surface, the inner surfaces being in communication with the fluid conducting structure and thus adapted to get in contact with a fluid in the fluid conducting structure, and (b) a vented enclosure in which the outer surfaces of the permeable means are arranged, wherein an initial RH in the range 20-40% in the enclosure can be raised at least 20%-point by transport of water vapour through the first permeable means when a sufficient amount of water is in contact with the inner surface thereof, this reducing the partial pressure of air in the enclosure and thus the pressure difference of air across the second permeable means. Strictly speaking the first means of the invention are permeable to water molecules and may, depending upon the conditions on either side thereof, allow water vapour to be generated from the second surface, however, in the context of the present invention such a membrane is characterized as a water vapour permeable membrane as its second surface is vented to the atmosphere and thus allowed to “generate” water vapour.

By raising the RH in the enclosure for a fluidic system as described above, a system is provided which will aid in expelling bubbles from a fluid assembly, once they are there, as well as reducing the likelihood of air entering into the fluid assembly. Further, the water vapour permeable means provides a simple means for humidifying the atmosphere on the other side of this membrane if the ambient atmosphere is not already humid. The fluidic system (or device) of the invention could be provided as a stand-alone unit to be used in combination with e.g. a fluid transport device, a sensor or a filter, or it could be provided as an integrated part of such a device or system.

The above definition of the ability to raise the RH at least 20%-point when the initial RH in the enclosure is in the range 20-40% can be considered a minimum requirement for the system. However, a given system fulfilling this requirement may indeed be able to raise the RH at least 20%-point also when the initial RH in the enclosure is in the range 40-60%. If the initial RH is e.g. 70 the system may still be able to raise the RH with a useful 15%-point or more. Advantageously the system is designed to achieve the specified raise in the temperature with a constant or varying temperature in the range 15-40° C., e.g. at a constant temperature of 20 or 37° C. The time to reach the defined raise will depend on a number of properties as well as the intended use, e.g. for some applications a fast raise may be desirable (e.g. in less than an hour) whereas for other applications it may be acceptable if the desired raise is achieved within 4, 8 or 12 hours. Properties that will influence the performance of the system are e.g. the volume of the enclosure, the size of the vent, the area and properties of the first permeable means, the volume of the fluid-conducting structure, and the flow rate (including zero) of liquid there through. However, the election of these properties and parameters to achieve the desired performance of the system can be considered an object of a normal design procedure. For example, a given fluidic system may be adapted to operate at a given flow rate in an exterior atmosphere having a RH in the range 20-50%, and wherein the raise in RH of at least 20%-point is established in the enclosure in less than 4 hours. The structure, properties and dimensions of such a system and its components could vary considerably.

The enclosure may comprise a vent towards the exterior atmosphere allowing a flow of water vapour to be established between the first permeable means and the vent. In this way a relatively constant atmosphere can be created in the enclosure by simple means.

The first and second permeable means may be in the form of a common member having common inner and outer surfaces, e.g. a moulded silicone rubber membrane.

In an exemplary embodiment the fluidic system comprises a pump arrangement adapted to provide a flow of fluid through the fluid conducting structure from the inlet to the outlet and thereby a flow of fluid past the inner surface of the first permeable means. The system may be provided with an actuator for actuating the pump arrangement, as well as a transcutaneous device adapted to be inserted through the skin of a subject, the transcutaneous device being arranged or adapted to be arranged in fluid communication with the outlet.

In a second aspect a method of operating a fluidic system is provided, comprising the steps of (a) providing a fluid assembly comprising (i) a fluid-conducting structure having an inlet and an outlet, (ii) first means being permeable to water vapour, and (iii) second means being permeable to air, wherein the first and second permeable means have an inner and an outer surface, the inner surfaces being in communication with the fluid conducting structure and thus adapted to get in contact with a fluid in the fluid conducting structure, (b) providing a vented enclosure in which the outer surfaces of the permeable means are arranged, the enclosure having an initial RH in the range 20-40%, and (c) raising the initial RH in the enclosure at least 20%-point by transport of water vapour through the first permeable means, this reducing the partial pressure of air in the enclosure and thus the pressure difference of air across the second permeable means.

In respect of the above-described method the same general considerations apply as discussed above in respect of the corresponding fluidic system. The fluidic system may thus be operated between an initial state in which the inner surface of the first permeable means are not in contact with water, and an operational state in which the inner surface of the first permeable means are in contact with water. The method may comprise the further step of establishing a flow of water-containing fluid through the fluid-conducting structure, the water-containing fluid being in fluid communication with the inner surface of the first permeable means. In the initial state the fluid-conducting structure may be essentially free from water. The enclosure may comprise a vent towards the exterior atmosphere allowing a flow of water vapour to be established between the first permeable means and the vent. The fluid assembly may be a pump assembly adapted to provide a flow of fluid through the fluid conducting structure from the inlet to the outlet, and the first and second permeable means may be in the form of a common member having common inner and outer surfaces.

In a further more general aspect a method of operating a fluidic system in an atmosphere comprising a given gas is provided, the method comprising the steps of (a) providing a fluid assembly comprising (i) a fluid-conducting structure having an inlet and an outlet, (ii) first means being permeable to the given fluid, and (iii) second means being permeable to the given gas, wherein the first and second permeable means have an inner and an outer surface, the inner surfaces being in communication with the fluid conducting structure, (b) providing a vented enclosure in which the outer surfaces of the permeable means are arranged, (c) providing a fluid in fluid communication with the inner surface of the first permeable means e.g. by means of a flow of fluid, and (d) raising in the enclosure the partial pressure of the given fluid by transport thereof through the first permeable means, this reducing the partial pressure of the given gas in the enclosure and thus the pressure difference of the given gas across the second permeable means, thereby influencing the transport of the given gas through the second permeable means.

By raising the partial pressure of the given fluid in the enclosure for a fluid assembly as described above, a method is provided which will aid in expelling the given gas from a fluid assembly, as well as reducing the likelihood of the gas entering into the fluid assembly. Further, the fluid permeable means provides a simple means for humidifying the atmosphere on the other side of this membrane if the ambient atmosphere is not already humid, this in contrast to known concepts in which a diffusion gradient across a membrane is established actively, e.g. by conducting a flow of a gas across the outside of a permeable membrane, see e.g. U.S. Pat. No. 5,149,340, U.S. Pat. No. 7,097,690, U.S. Pat. No. 4,788,556 and U.S. Pat. No. 6,060,319.

The first permeable means may be permeable to vapour of the given fluid. The first and second permeable means are in the form of a common permeable member (e.g. a membrane) having common inner and outer surfaces.

In a yet further aspect of the invention a pump assembly is provided, comprising a fluid inlet and a fluid outlet, a suction pump having a pump inlet in fluid communication with the fluid inlet and a pump outlet in fluid communication with the fluid outlet, and a safety valve arranged between the fluid inlet and the fluid outlet. The safety valve comprises a first moveable portion (e.g. a flexible membrane) in flow communication with the fluid inlet, the first moveable portion having an initial state during operation of the suction pump, and an activated state when a positive pressure is applied to the fluid inlet, a second moveable portion (e.g. a flexible membrane) in flow communication with the fluid outlet, the second moveable portion having an initial state in which a flow of fluid to the fluid outlet is allowed, and an activated state in which a flow of fluid to the fluid outlet is prevented, and a moveable transmission member arranged between the first and second moveable portions and adapted to transmit movement there between. In this arrangement movement of the first moveable portion from the initial to the activated state results in the second moveable portion being moved from the initial to the activated state via the moveable transmission member, whereby a positive pressure applied to the fluid inlet will prevent a flow of fluid to the fluid outlet. The two moveable portions may be identical in respect of their pressure characteristics, however, as there will be a pressure drop across the suction pump, this drop will ensure will ensure that a raise in pressure in the inlet will result in closure of the safety valve.

By providing a safety valve with a “slave” secondary membrane, a valve is provided having two layers instead of one, this providing in a simple way (e.g. without using laminated membranes) a high degree of safety in case of rupture of one membrane (or leakage of a moveable portion).

Alternatively a pump assembly is provided comprising a fluid inlet and a fluid outlet, a suction pump having a pump inlet in fluid communication with the fluid inlet and a pump outlet in fluid communication with the fluid outlet, and a safety valve arranged between the fluid inlet and the fluid outlet. The safety valve comprises a primary membrane moved to an actuated position when a positive pressure is applied to the fluid inlet, a transmission member moved to an actuated position when the primary membrane is moved to an actuated position, a secondary membrane moved to an activated state when the transmission member is moved to its actuated position.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIG. 1A shows in schematic form a prior art arrangement for removal of air bubbles,

FIG. 1B shows in schematic form an arrangement for removal of air bubbles in accordance with the present invention,

FIG. 2 shows a schematic overview of a pump assembly connected to a reservoir,

FIGS. 3A and 3B show exploded views of a pump assembly,

FIG. 4 shows a cross-sectional view of the pump assembly of FIG. 3A in an assembled state,

FIGS. 5 and 6 show the exploded views of FIGS. 3A and 3B with the flow path indicated,

FIG. 7 shows a schematic representation of an embodiment of the invention,

FIGS. 8, 9, 11 and 12 show parameter examples for an embodiment of the invention,

FIG. 10 shows a graph illustrating pressure in a bubble vs. bubble radius, and

FIG. 13 shows a graph illustrating an example of RH build-up as a function of time for a fluidic system encompassing the present invention.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

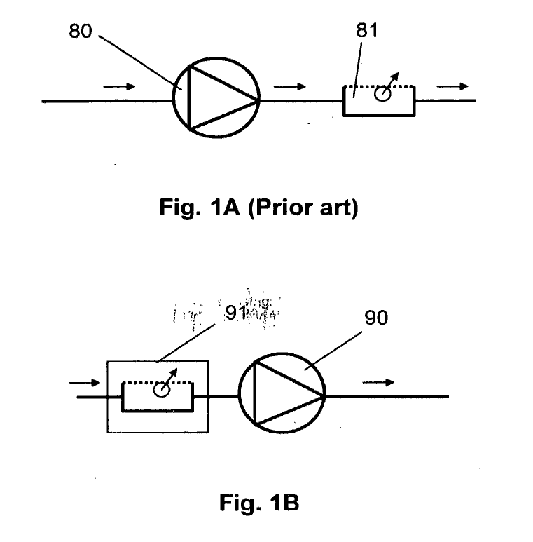

FIG. 1A shows a schematic representation of a prior art fluidic system adapted for removal of air bubbles, the system comprising a pump 80 and a down-stream bubble removal unit 81 provided with a hydrophobic vent, e.g. a Gore-Tex® membrane placed in the flow path. As appears, this arrangement will work only if the pressure inside the fluid path is higher than the ambient air pressure.

However, bubbles may also be an issue in a part of a fluidic system in which the pressure inside the fluid path is not higher than the ambient air pressure, i.e. the same or even lower. Correspondingly, a simple and reliable arrangement 91 for removal of air bubbles as illustrated schematically in FIG. 1B would thus be desirable for systems in which air bubbles would be detrimental to the functionality of a given component. In FIG. 1B the component is shown as a pump 90, however, it could also be in the form of a filter or a sensor unit. Indeed, the arrangement should also prevent air from entering the system. Before turning to the general principles of the present invention an implementation of the invention in the form of a pump system will be described.

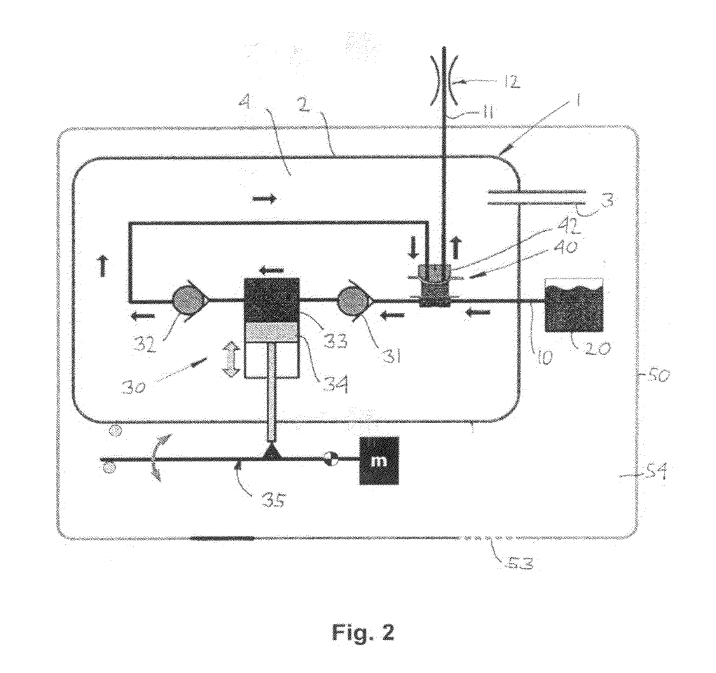

With reference to FIG. 2 a schematic overview of a pump system 1 connected to a reservoir 20 is shown, the pump system comprising the following general features: a fluid inlet 10 in fluid communication with the reservoir 20, a suction pump 30 per se having inlet and outlet valves 31, 32 and a pump chamber 33 with an associated piston 34 driven by an actuator 35, an outlet 11 connected to e.g. an infusion patch 12, and a combined safety valve 40. The combined safety valve has a primary side with the pressure in the inlet 10 acting on a piston 41 which again acts on an anti-suction membrane valve 42, this valve allowing a positive-pressure flow of fluid across the valve but does not allow a flow of fluid due to suction, e.g. as may be applied to outlet 11. The arrows indicate the flow direction between the individual components. The pump system further comprises a housing 2 with a vent 3, this establishing a vented enclosure 4 in which the above-described components (apart from the reservoir) are arranged. In the shown embodiment an outer housing 50 comprising a second vent 53 (e.g. in the form of a Gore-Tex® membrane) is provided, this establishing a vented enclosure 54 for the pump assembly.

When the piston is moved downwards (in the drawing) a relative negative pressure will build up inside the pump chamber which will cause the inlet valve to open and subsequently fluid will be drawn form the reservoir through the open primary side of the safety valve by suction action. When the piston is moved upwards (in the drawing) a relative overpressure will build up in the pump chamber which will cause the inlet valve to close and the outlet valve and the safety valve to open whereby fluid will flow from the pump chamber through the outlet valve and the secondary side of the safety valve to the outlet. As appears, in normal operation the combined safety valve allows fluid passage during both intake and expelling of fluid and is thus “passive” during normal operation. However, in case the reservoir is pressurized (as may happen for a flexible reservoir) the elevated pressure in the reservoir will be transmitted to both the primary side of the safety valve and, via the pump chamber, the secondary side of the safety valve in which case the pressure on the primary side of the safety valve will prevent the secondary side to open due to e.g. the pressure drop across the inlet and outlet valves.

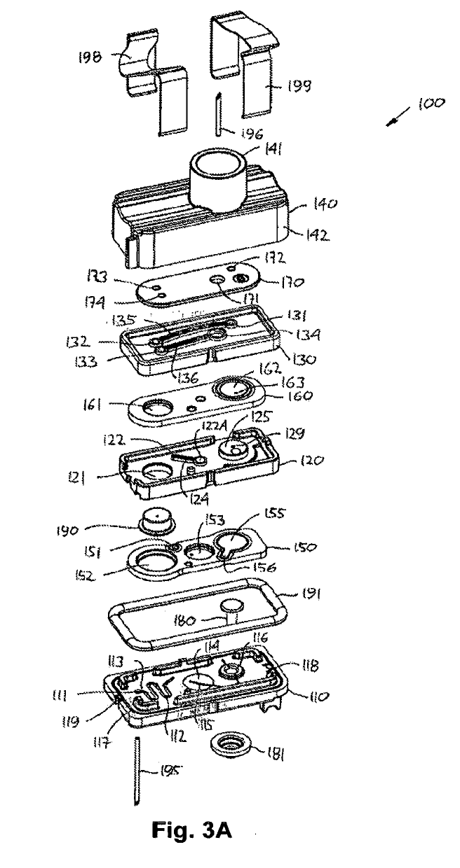

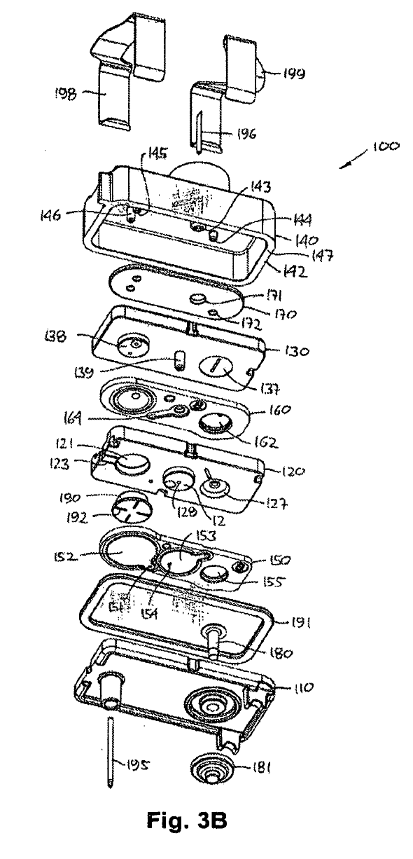

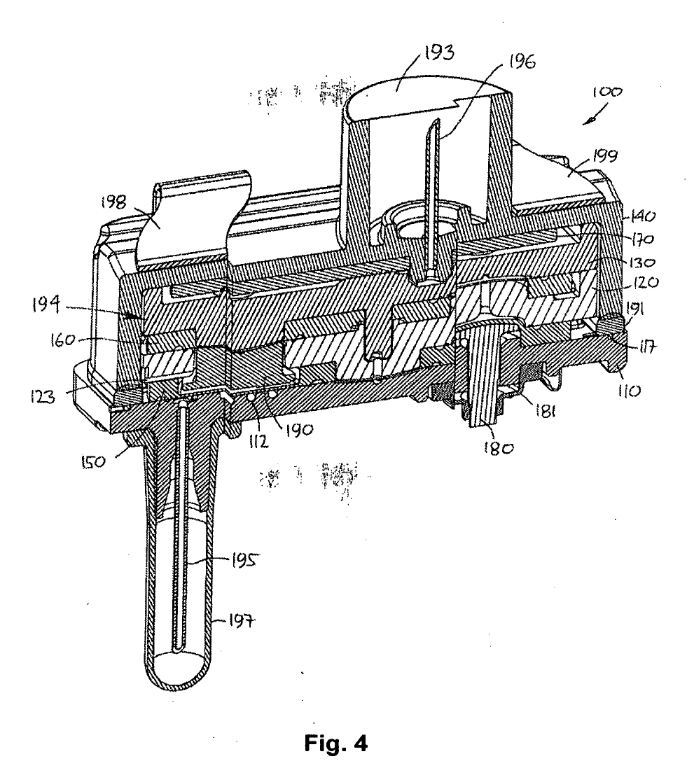

In FIGS. 3A and 3B an exploded view (seen from above respectively below) of a pump system 100 utilizing the pump principle depicted in FIG. 2 is shown, the pump system being suitable for use with e.g. a flexible reservoir. The system comprises a pump assembly (i.e. a pump per se) with an integrated housing. The pump is a membrane pump comprising a piston-actuated pump membrane with flow-controlled inlet- and outlet-valves. The pump has a general layered construction comprising rigid plates in the form of a bottom plate 110, a middle plate 120, a top plate B 130, and a top plate A 140 between which are interposed flexible membrane members in the form of (from below) a second membrane 150, a first membrane 160, and a third membrane 170. The pump further comprises a piston 180 interposed between the bottom plate and the second membrane, a piston gasket 181 arranged between the piston stem and the bottom plate, a safety valve piston 190 arranged in the middle plate and interposed between the first and second membrane, a main gasket 191 interposed between the skirt 142 of top plate A and the bottom plate, and inlet and outlet conduits 195, 196, here in the form of pointed hollow needles. The layers are held in a stacked arrangement by outer clips 198, 199. The pump is supplied to a user in a sterile state with a needle penetratable tubular elastomeric sealing member 197 covering the inlet needle 195 and a penetratable paper seal 193 (see FIG. 4) covering the outlet conduit. This design allows the tubular sealing member to be penetrated and collapse when the needle 197 is pushed into engagement with a fluid source, e.g. a drug reservoir.

Next the different functional components of the individual members will be described with reference to FIGS. 3A and 3B, the members having an “upper” surface facing in direction of the outlet and a “lower” surface facing in direction of the inlet. In general the different valves each comprise a valve seat across which a first surface of a flexible valve membrane is arranged, a valve cavity being formed between the second surface of the valve membrane and an opposed valve wall or valve “roof”. Depending on the function of the valve, openings may be formed in the valve seat and valve membrane. Apart from the primary side safety valve membrane, all the valve membranes are tensioned against the corresponding valve seat thus requiring a given pressure differential across the valve in order to open. The top plates comprise a number of cylindrical core members with an outer channel along there length, however, these core members are only provided for the cost-effective manufacture of fine bores in the members through which they are arranged.

The bottom plate 110 comprises an upper surface with an inlet bore 111 in flow communication with a serpentine channel 112 arranged across a first safety valve seat 113, an inlet valve wall 114 with a transfer channel 115, a piston bore 116 for the piston stem, an open circumferential channel 117 having an inlet channel 118 and an opposed outlet 119, and on the lower surface mounting means for an actuator.

The second membrane 150 comprises a bore 151, a primary side safety valve membrane 152, an inlet valve membrane 153 with an opening 154, and a pump membrane 155 in communication with a bore 156.

The middle plate 120 comprises a piston bore 121 for the safety valve piston 190, first and second bores 122, 122A, an upper surface with a transfer channel 124 interconnecting the first and second bores, and an outlet valve seat 125, a lover surface with an inlet valve seat 126, a pump cavity 127, and a pair of vent channels 123 between the piston bore and the exterior. The inlet valve seat comprises an opening 128 in communication with the second bore 122A, just as a bore 129 connects the pump cavity and the outlet valve seat 125.

The first membrane 160 comprises a secondary side safety valve membrane 161, an outlet valve membrane 162 with an opening 163, an opening for a core member 139, and a lover surface with a channel 164 adapted to engage the transfer channel 124.

The top plate B 130 comprises first, second and third bores 131, 132, 133 as well as partial bore 134, an upper surface with a curved first transfer channel 135 interconnecting the first and second bores, and a straight second transfer channel 136 interconnecting the third bore and the partial bore, a lover surface with an outlet valve wall 137 having an opening in flow communication with the first bore 131, a second safety valve seat 138 having first and second openings in flow communications with the second respectively third bores 132, 133, and a core member 139 adapted to engage the middle plate 120.

The third membrane 170 comprises an outlet bore 171 adapted to receive a core member 143, three openings 172, 173, 174 for core members 144, 145, 146, and a substantially planar lower surface adapted to engage the first and second channels in the top plate B.

The top plate A 140 comprises an outlet bore adapted to receive the outlet tube 196, an upper surface with a cylindrical member 141 surrounding the outlet tube, a lower surface with a circumferential skirt 142 having a circumferential lower edge 147, a first core member 143 comprising the outlet bore and adapted to be received in the partial bore 134 of the top plate A, and three further core members 144, 145, 146 adapted to be received in the bores 131, 132, 133 of the top plate B.

FIG. 4 shows a cross-sectional view of the pump system 100 of FIG. 3A in an assembled stacked state in which the four plates 110, 120, 130, 140, the three membranes 150, 160, 170, the piston 180, the safety valve piston 190 and the main gasket 191 can be seen together with many of the above-described structures. The circumferential lower edge 147 of the skirt 142 engages the upper surface of the bottom plate with the main gasket 191 interposed there between, this establishing an enclosure 194 for the remaining elements stacked between the bottom plate and the top plate A. As appears, apart from a narrow circumferential gap, the enclosed stacked elements almost occupy the enclosure. As also appears, the main gasket engages the circumferential channel 117 in the bottom plate and thus establishes a closed circumferential channel with an inlet channel 118 and an opposed outlet 119, this allowing the channel to serve as a vent. In the shown embodiment the housing is formed integrally with the bottom plate and the top plate A, however, the housing may also be provided as a separate structure.

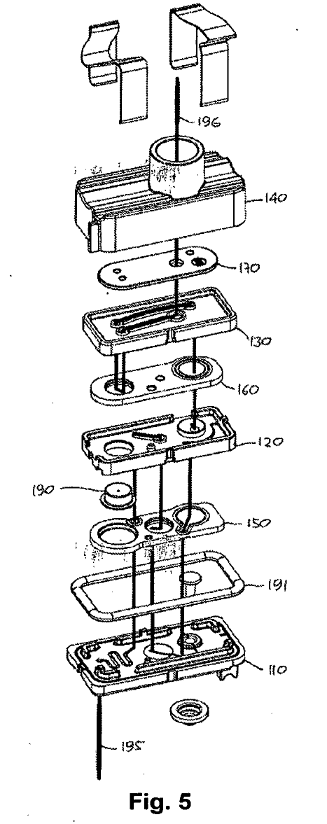

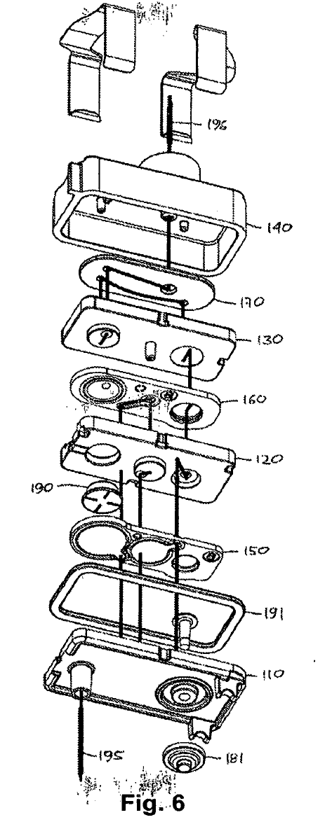

With reference to FIGS. 5 and 6 the flow path through the pump assembly will be described. FIGS. 5 and 6 essentially correspond to FIGS. 3A and 3B but with the flow path shown schematically. It should be noted that the shown flow path differs in the two figures as it has been drawn to illustrate flow across the surfaces actually shown, i.e. in FIG. 5 the flow path is shown corresponding to the upper surfaces and in FIG. 6 the flow path is shown corresponding to the lower surfaces.

Thus, fluid will enter (i.e. sucked into) the pump assembly 100 through the inlet tube 195 and inlet bore 111, cross the first safety valve seat 113 along the serpentine channel 112 and enter the bores 151, 122 in the second membrane respectively the middle plate, flow through the transfer channel 124 to the inlet valve seat 126 via opening 128 where it crosses the valve seat and flows through the opening 154 in the inlet valve membrane 153. From the inlet valve the fluid will flow across the valve wall 114 along the transfer channel 115 and through bore 156 of the pump membrane 155 to the pump chamber 127 from where it will be pumped through the bore 129 to the outlet valve seat 125. The fluid will then cross the outlet valve seat and be forced through the opening 163 in the outlet valve membrane to the curved first transfer channel 135 via bore 131. The fluid will then cross the second safety valve seat 138 via bores 132, 133 and enter the straight second transfer channel 136 from where it will leave the pump assembly through the outlet bore of core member 143 and outlet tube 196.

In normal operation the primary side safety valve membrane 152 will rest against the first safety valve seat 113 and the fluid will flow along the serpentine channel 112 without lifting the valve membrane. On the secondary side the secondary side safety valve membrane 161 will be lifted from the valve seat 138 as the fluid crosses from the first to the second transfer channel 135, 136 in top plate B. In case the fluid in the inlet is pressurized the primary side safety valve membrane will be lifted from its seat and move the safety piston 190 upwards against the secondary side safety valve membrane and thus close the secondary side safety valve. In principle the pressure should be the same on the two safety valve membranes, however, due to the pressure drop across the inlet and outlet valves as well as the opening pressure necessary to overcome the flow resistance of the pre-tensioned secondary side valve membrane, the pressure acting on the primary side of the safety piston will be higher than the pressure acting on its secondary side, this resulting in a closed safety valve. As also appears, in case suction is applied to the outlet side, this will close flow across the secondary side of the safety valve.

As described above with reference to FIGS. 2 and 4, the pump system comprises a housing with a vent, this establishing a vented enclosure for the pump per se. The main purpose of the vented housing is to create, in cooperation with one or more permeable membrane portions of the pump, a high RH micro-climate around the pump. In the shown embodiment all three membranes are made from the same elastomeric material (e.g. silicone rubber) being permeable to both water vapour and air which means that there will be a transport of water vapour from a water containing fluid in the flow channel to the enclosure through all parts of the membranes exposed to the atmosphere in the enclosure, however, in practise the largest amount of water will penetrate through the portions of the membranes which are both large and thin, which for the shown embodiment will mean the primary and secondary safety valve membranes, especially the primary due to its contact with the serpentine channel 112. It is to be noticed that the piston gasket 181 is arranged outside the enclosure. Correspondingly, this gasket should be made from a vapour and gas tight material or a small loss of water there through should be acceptable. Water vapour and air is only used as an example as the present invention may be used also for other liquids and gases.

In the following a “main” path of water vapour originating from the primary safety valve membrane will be described. As described above the flow path through the pump comprises a serpentine channel 112 in contact with the lower surface of the primary safety valve membrane 152. This membrane is relatively thin and an amount of water vapour will penetrate the membrane and enter the space between the lower surface of the safety valve piston 190 and the upper surface of the primary safety valve membrane, this space being hold open by a number of protrusions 192 on the lower surface of the piston, see FIG. 3. From here and via the pair of vent channels 123 it will enter the narrow circumferential space 194 established between the circumferential skirt 142 and the elements stacked between the bottom plate and the top plate A. Via the outlet channel 118 the water vapour will enter the circumferential vent channel 117 from where it will leave the pump system through outlet 119. Correspondingly, water vapour will also enter the enclosure via the secondary safety valve membrane just as an amount of water vapour will penetrate through the outer circumferential portions of the membranes. Due to the relatively small volume inside the enclosure, the relatively high permeability of the primary safety valve membrane, as well as the long vent channel, it is possible to create a large RH differential between the interior of the enclosure and the exterior in a relatively short period of time. As will be explained in greater detail below, the high RH created inside the enclosure ensures that the loss of water from the fluid path as well as the amount of air in the fluid path are reduced. Indeed, the actual dimensions and other parameters for any given pump or other liquid system will determine the efficiency with which such an RH differential is established and maintained.

In the shown embodiment an enclosure is established for the entire stack of elements, this in order to enclose all exposed membrane surfaces, however, in alternative embodiments the enclosure may be smaller and only serve to enclose a smaller part of the pump. For example, the pump may be designed in such a way that there is essentially no transport of vapour through the outer surfaces of the membranes, e.g. by a coating or other constructional means. However, for such an embodiment the safety valve membranes would still need to be vented to the exterior and thus provide a source of water and air penetration. For such an embodiment the space created around the safety valve piston 190 (see FIG. 4) would establish an enclosure for the two safety membranes just as the vent could be created by a narrow channel established between the second membrane 150 and the middle plate 120.

In the above an example of a pump system implementing the present invention has been described. With reference to such a pump system principles of the present invention will be exemplified and explained in greater detail.

Air bubbles are often a problem in fluidic systems such as pumps, especially when they are containing chambers interconnected by channels. Thus, when filling a system with liquid for the first time, it can be difficult to avoid enclosed air in the system, e.g. in case of dimensional changes of the fluid path. The enclosed air bubbles may then cause pressure losses when the bubble-filled liquid is transported through a fluid path.

Further, if the fluidic system contains highly permeable elements, e.g. made from silicone rubber, separating the fluid from the ambient atmosphere, air bubbles can also enter into the system by diffusion. This diffusion is driven by differences in partial pressure of the gasses prevalent inside and outside the fluidic system.

Bubble problems have traditionally been solved by (i) a hydrophobic vent, e.g. a Gore-Tex® membrane placed in the flow path, however, this will work only if the pressure inside the fluid path is higher than the ambient air pressure, or (ii) a bubble trap that collects the bubbles and thus prevents them from entering certain parts of the fluid path, however, as this does not eliminate the bubbles but only separates them from the liquid, this solution takes up a volume which may not be feasible to have in a given system, especially if new air will enter the system during operation. The drawbacks of both of these conventional methods for bubble elimination are solved by the present invention.

As illustrated above with reference to a pump system, the present invention (i) integrates a permeable membrane, e.g. a silicone rubber membrane, into a fluidic system in such a way that the fluid gets in contact with one side of the membrane, and (ii) provides means for humidifying the atmosphere on the other side of this membrane if the ambient atmosphere is not already humid, e.g. by putting the system into a box which will then serve to create a humid atmosphere inside by means of the membrane, see FIG. 7 which schematically shows an embodiment of a fluidic system in the form of an air bubble removal unit as also shown in FIG. 2B.

By these two features the problems associated with bubbles in a fluidic system can be reduced and potentially avoided. The method allows elimination of bubbles from a fluidic system, not only from areas with over-pressure, but also areas with neutral pressure, and even with a slight under-pressure.

The present invention thus has two main aspects as will be illustrated by the following two examples: (1) a method to avoid air from entering into a fluidic system by diffusion, and (2) a method to expel bubbles from a fluidic system, once they are there.

### EXAMPLE 1

### Avoiding Gas from Entering the System by Diffusion

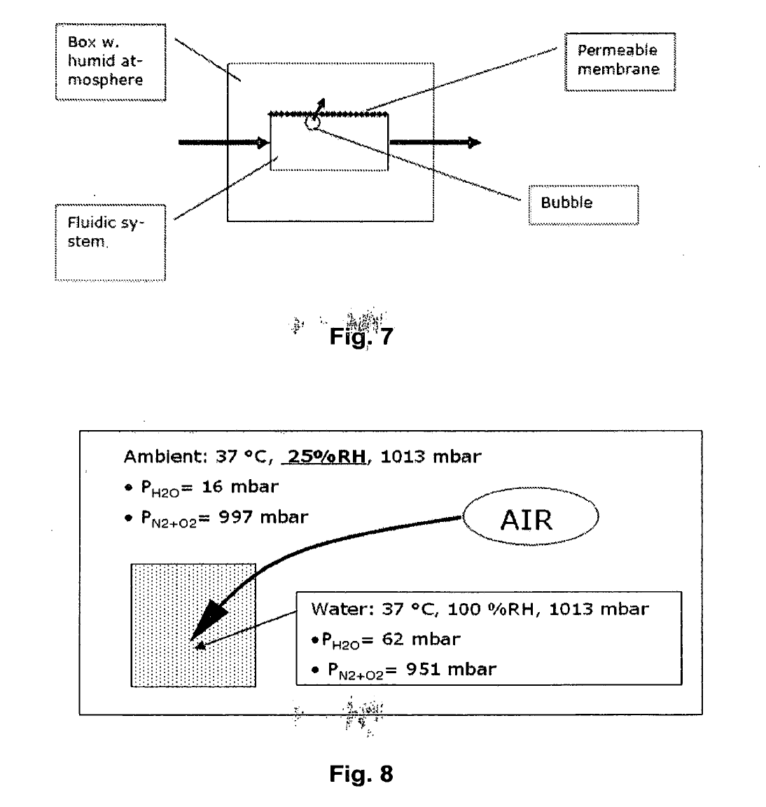

First, a transport mechanism in an example where the fluid is water, and the ambient atmosphere is air will be described. FIG. 8 shows the gas transport mechanism in a 37° C. warm water-filled fluidic system exposed to 37° C., 25% RH atmospheric air (˜79% N₂ and 21% O₂). According to Dalton's law the total pressure in a gas mixture equals the sum of the individual gasses' partial pressures. This results in a lower pressure of air, P_{N2+O2}, inside the fluidic system than outside as the total pressure is the same inside and outside, and the water vapour pressure is higher inside the system. This difference in P_{N2+O2} will try to drive air into the system.

A basic principle of the present invention is to provide the fluidic system with an outer shield. The surprising part is that this shield does not have to be gas tight in order to maintain a humid atmosphere around the fluidic system. In FIG. 9 below a box is drawn around the fluidic system symbolising an outer shield that equalizes P_{N2+O2} inside and outside the fluidic system and stops the air transport, this preventing air from diffusing into the system through the membrane.

### EXAMPLE 2

### Expelling Bubbles from a Fluidic System

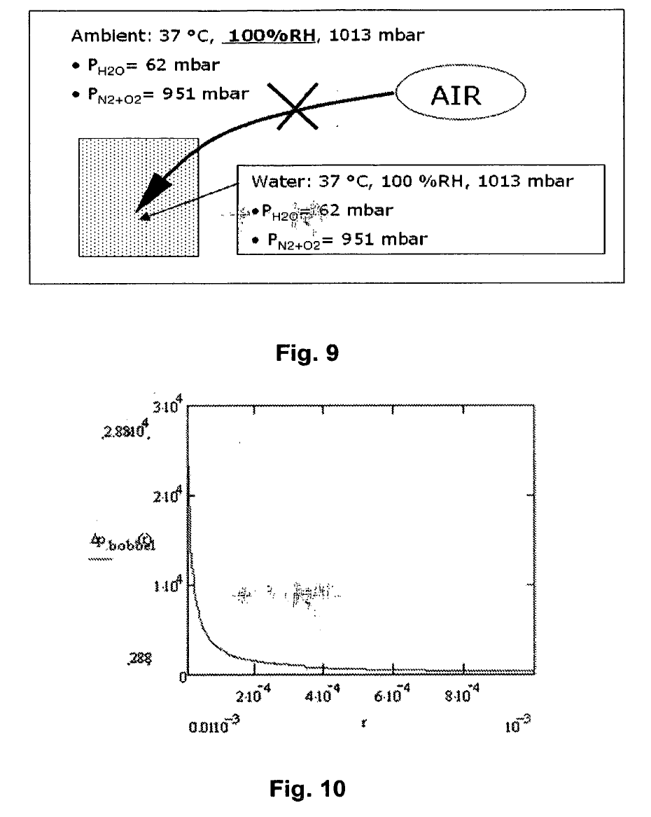

This aspect provides a method to eliminate bubbles from a fluidic system once they have been introduced. It works in areas with over-pressure, neutral pressure, and even areas with a slight under-pressure. This aspect of the present invention is based on the fact that the surface tension of the liquid will make small bubbles act as pressure tanks. In the graph shown in FIG. 10 the internal over-pressure in a bubble vs. bubble radius in water is illustrated. According to the graph a bubble with a diameter of e.g. 300 μm will have an internal over-pressure of approximately 30 mbar which is a realistic bubble size in a fluidic system.

When this phenomenon is added to the transport mechanism a highly surprising effect will show: Even if the shield described above does not establish 100% RH it can prevent air from entering the fluidic system and even expel bubbles from the system. This is explained in the following two examples.

### EXAMPLE 2A

### Ambient dry atmosphere (37° C. 25% RH)

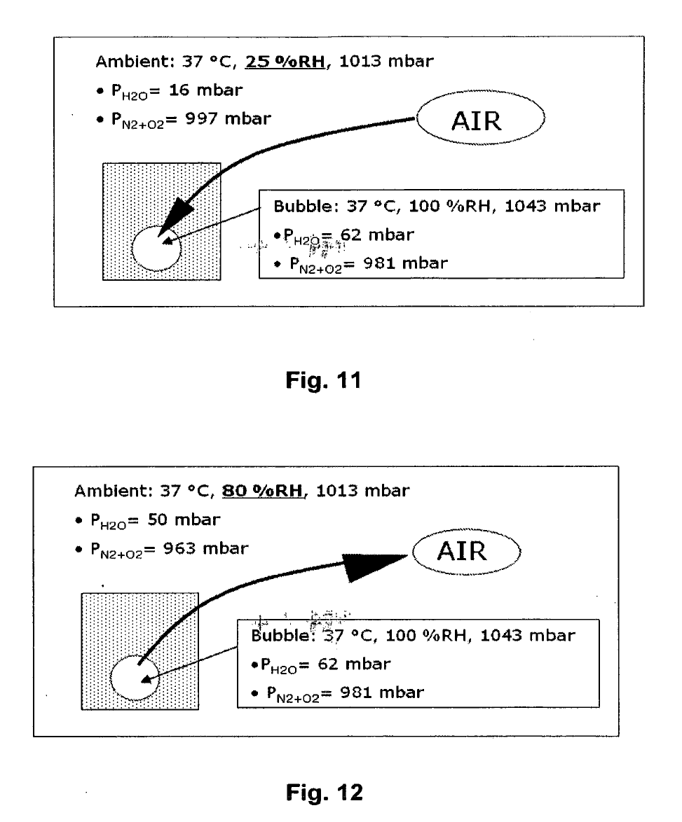

The following statements apply for the conditions in a system as shown in FIG. 11: (i) High pressure of air (N₂+O₂) in the ambient atmosphere because the water vapour is only occupying a very small part of the total pressure, (ii) Because of the moist atmosphere inside the bubble, a bubble pressure of e.g. 30 mbar will still result in a lower air pressure in the bubble in the atmosphere, (iii) As a result air will diffuse into the bubble.

### EXAMPLE 2B

### Ambient Moist Atmosphere (37° C. 80% RH)

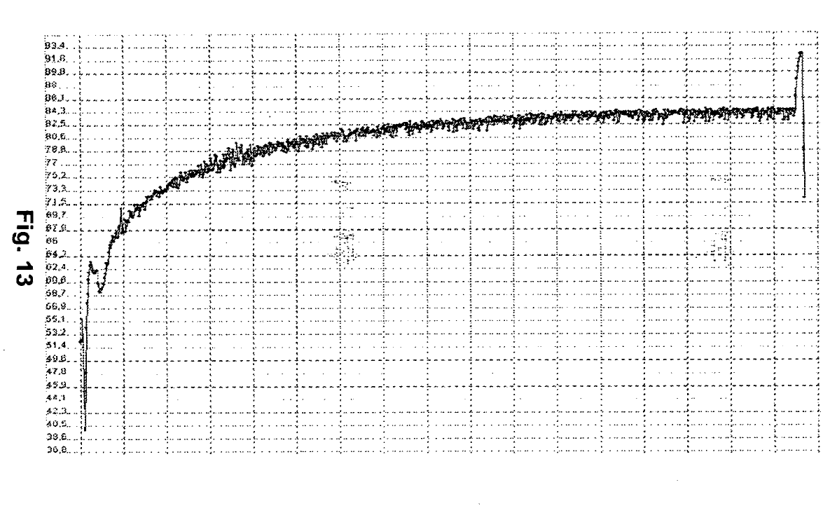

The following statements apply for the conditions in a system as shown in FIG. 12: (i) Lower pressure of air (N₂+O₂) in the ambient atmosphere because water vapour is occupying a larger part of the total pressure, (ii) The surface tension of the bubble will cause the air pressure inside the bubble to be higher than the ambient atmosphere, (iii) As a result air will diffuse out of the bubble.

### EXAMPLE 3

### Raising RH in Enclosure surrounding Miniature Pump Assembly

A miniature pump assembly of the type shown in FIGS. 3-6, i.e. comprising a vented enclosure and a safety valve membrane, was arranged in an ambient dry atmosphere (37° C., 25% RH) with a miniature moisture sensor arranged in the enclosure. The silicone rubber safety valve membrane had a diameter of 6 mm and a thickness of 0.1 mm, this membrane providing the majority of the diffusion area between the pump flow path and the enclosure. After the flow path having a volume of 18 μl mm3 had been initially primed with water the pump was operated with a flow rate of 1 μl/hr. The resulting raise in RH (at 37° C.) as a function of time (x-axis divided in units of 1 hr) is shown in FIG. 13. As appears, the combination of the area of the diffusion membrane, the volume of the enclosure (75 μl mm³) and the actual flow rate through the pump was sufficient to build up and sustain a high RH. More specifically, after 2 hr a RH of approx. 73% RH had been reached in the enclosure, after 4 hours a RH of approx. 80% RH had been reached in the enclosure, after 8 hours a RH of approx. 82% RH had been reached in the enclosure, and after 16 hours a RH of approx. 84% RH had been reached in the enclosure. It was further observed that the majority of bubbles introduced during the priming disappeared and that no new bubbles developed during the experiment.

The above-described pump assembly may be provided in a drug delivery device of the type shown in e.g. EP 1 527 792 or WO 2006/077263, which is hereby incorporated by reference. In a situation of use where the reservoir unit is attached to a transcutaneous device unit the outlet tube 196 is connected to an inlet of the transcutaneous device unit, and the inlet tube 195 is connected to a flexible reservoir allowing a fluid to be sucked into the flow path of the pump. The tubes may be pointed or blunt and adapted to be inserted through a corresponding septum.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A fluidic system comprising:
a) a fluid assembly (100) comprising:
(i) a fluid-conducting structure (112) having an inlet (195) and an outlet (196),
(ii) first means being permeable to water vapour, and
(iii) second means being permeable to air,
wherein the first and second permeable means have an inner and an outer surface, the inner surfaces being in communication with the fluid conducting structure and thus adapted to get in contact with a fluid in the fluid conducting structure, and
b) a vented enclosure (194) in which the outer surfaces of the permeable means are arranged,
wherein an initial RH in the range 20-40% in the enclosure can be raised at least 20%-point by transport of water vapour through the first permeable means when a sufficient amount of water is in contact with the inner surface thereof, this reducing the partial pressure of air in the enclosure and thus the pressure difference of air across the second permeable means.

2. A fluidic system as in claim 1, adapted to operate in an exterior atmosphere having a RH in the range 20-50%, and wherein the raise in RH of at least 20%-point is established in the enclosure in less than 4 hours.

3. A fluidic system as in claim 1, wherein the enclosure comprises a vent (119) towards the exterior atmosphere allowing a flow of water vapour to be established between the first permeable means and the vent.

4. A fluidic system as in claim 1, wherein the first and second permeable means are in the form of a common member (152) having common inner and outer surfaces.

5. A fluidic system as in claim 1, wherein the fluid assembly comprises a pump arrangement adapted to provide a flow of fluid through the fluid conducting structure from the inlet to the outlet and thereby a flow of fluid past the inner surface of the first permeable means.

6. A fluidic system as in claim 1, further comprising an actuator for actuating the pump arrangement, and a transcutaneous device adapted to be inserted through the skin of a subject, the transcutaneous device being arranged or adapted to be arranged in fluid communication with the outlet.

7. A method of operating a fluidic system, comprising the steps of:
a) providing a fluid assembly comprising:
(i) a fluid-conducting structure having an inlet and an outlet,
(ii) first means being permeable to water vapour, and
(iii) second means being permeable to air,
wherein the first and second permeable means have an inner and an outer surface, the inner surfaces being in communication with the fluid conducting structure and thus adapted to get in contact with a fluid in the fluid conducting structure,
b) providing a vented enclosure in which the outer surfaces of the permeable means are arranged, the enclosure having an initial RH in the range 20-40%, and
c) raising the initial RH in the enclosure at least 20%-point by transport of water vapour through the first permeable means, this reducing the partial pressure of air in the enclosure and thus the pressure difference of air across the second permeable means.

8. A method as in claim 7, wherein the fluidic system is operated between an initial state in which the inner surface of the first permeable means are not in contact with water, and an operational state in which the inner surface of the first permeable means are in contact with water.

9. A method as in claim 7, comprising the further step of establishing a flow of water-containing fluid through the fluid-conducting structure, the water-containing fluid being in fluid communication with the inner surface of the first permeable means.

10. A method as in claim 7, wherein in the initial state the fluid-conducting structure is essentially free from water.

11. A method as in claim 7, wherein the fluidic system is operated in an exterior atmosphere having a RH in the range 20-50%, and wherein the raise in RH of at least 20%-point is established in the enclosure in less than 4 hours.

12. A method as in claim 7, wherein the enclosure comprises a vent towards the exterior atmosphere allowing a flow of water vapour to be established between the first permeable means and the vent.

13. A method as in claim 7, wherein the fluid assembly is a pump assembly adapted to provide a flow of fluid through the fluid conducting structure from the inlet to the outlet.

14. A method as in claim 7, wherein the first and second permeable means are in the form of a common member having common inner and outer surfaces.

15. A method of operating a fluidic system in an atmosphere comprising a given gas, the method comprising the steps of:
a) providing a fluid assembly comprising:
(i) a fluid-conducting structure having an inlet and an outlet,
(ii) first means being permeable to the given fluid, and
(iii) second means being permeable to the given gas,
wherein the first and second permeable means have an inner and an outer surface, the inner surfaces being in communication with the fluid conducting structure,
b) providing a vented enclosure in which the outer surfaces of the permeable means are arranged,
c) providing a fluid in fluid communication with the inner surface of the first permeable means, and
d) raising in the enclosure the partial pressure of the given fluid by transport thereof through the first permeable means, this reducing the partial pressure of the given gas in the enclosure and thus the pressure difference of the given gas across the second permeable means, thereby influencing the transport of the given gas through the second permeable means.

16. A method as in claim 15, wherein the first permeable means is permeable to vapour of the given fluid.

17. A method as in claim 15, wherein the first and second permeable means are in the form of a common permeable member having common inner and outer surfaces.

18. A method as in claim 15, wherein a flow of fluid is established through the fluid-conducting structure of the fluid assembly, the fluid being in fluid communication with the inner surfaces of the first permeable means.
